(19) United States
(12) Patent Application Publication
(10) Pub. No.: US 2025/0200214 A1
(43) Pub. Date: Jun. 19, 2025

(54) Framework for secure on-site patient level analytics

(71) Applicant: Clinerion Ltd., Basel (CH)

(72) Inventors: Matteo BERCHIER, Poschiavo (CH); Andreas WALTER, Baden-Baden (DE); Bernhard BODENMANN, Binningen (CH)

(21) Appl. No.: 18/545,253

(22) Filed: Dec. 19, 2023

(30) Foreign Application Priority Data
Dec. 18, 2023 (EP) 23217665.1

(51) Int. Cl. G06F 21/62; G16H 10/60

(52) U.S. Cl. CPC G06F 21/6245; G16H 10/60

(57) ABSTRACT

A data protection safeguarding analytics system for federated patient data, at least has a plurality of analytics environments which are separate from one another spatially as well as system-wise, which in each case include at last one local patient analytics database comprising a portion of the federated patient data, and which in each case include at least one local analytics module that is configured at least for an execution of scripts on the portion of the federated patient data available in the respective local patient analytics database, the data protection safeguarding analytics system further having at least one external operator and/or programming interface system, which is in particular arranged centrally or distributedly, which is separate from the analytics environments spatially and system-wise, by means of which the scripts that can be executed by the local analytics modules can be—in particular externally—created and/or provided, and which is with regard to data transfer connected to each of the analytics environments exclusively via connections, in particular outbound connections, that are initiated from the analytics environments.

### PRIOR ART

The invention concerns an analytics system according to claim 1, an analytics environment or an interface system according to claim 15 and an analytics method according to claim 16.

It has already been proposed to analyze patient data from hospital information systems for a variety of purposes, for example in order to identify test persons for clinical surveys or to analyze treatment success, etc. As the data stored in hospital information systems are particularly sensitive data worthy to be protected, data safety is crucial.

The objective of the invention is in particular to provide a generic device having advantageous properties regarding a data protection safeguarding analytics of patient data. The objective is achieved according to the invention by the features of the independent patent claims while advantageous implementations and further developments of the invention may be gathered from the subclaims.

### ADVANTAGES OF THE INVENTION

A data protection safeguarding analytics system for federated patient data is proposed, in particular for a recruiting of test persons for clinical surveys, for identifying trends, for obtaining optimization proposals for treatments, for an implementation of clinical dashboards and/or for a modeling of machine-learning methods, at least with a plurality of analytics environments which are separate from one another spatially as well as system-wise, which in each case include at least one local patient analytics database comprising a portion of the federated patient data and which in each case include at least one local analytics module that is configured at least for an execution of scripts on the portion of the federated patient data available in the respective local patient analytics database, and with at least one external operator and/or programming interface system, which is in particular arranged centrally or distributedly, which is separate from the analytics environments spatially as well as system-wise, by means of which the scripts that can be executed by the local analytics modules can be—in particular externally—created and/or provided, and which is with regard to data transfer connected to each of the analytics environments exclusively via connections, in particular so-called outbound connections, that are initiated from the analytics environments. This advantageously allows providing a data protection safeguarding analytics possibility based on the execution of scripts. It is advantageously possible to create an analytics possibility that is usable in especially multiple ways and at the same time has an especially high degree of data protection for sensitive patient data. Advantageously a possibility of local execution of externally created scripts can be provided for data analytics, which is based only on outbound connections and thus does not open external accessibility to the local patient analytics databases. This advantageously allows achieving that an installation of the analytics system cannot have any negative influence on the data safety of the hospital information systems comprising the local patient analytics databases. The proposed analytics system advantageously provides the possibility of applying externally prepared scripts to patient data from hospital information systems, which are only locally available and are especially sensitive, without enabling data leakage by which sensitive information could leave the local hospital information systems. Advantageously the proposed analytics system creates by means of the proposed system architecture neither exploiting nor hacking possibilities by which unauthorized access could be enabled to the local systems, like the hospital information systems. In particular, for a maximization of a safety of the analytics system, all scripts of the analytics system which are meant to be executable by the local analytics modules are subjected to a manual or automatized safety check/a manual or automatized script check. This advantageously allows excluding safety leakage caused by the scripts. Advantageously a high degree of data safety is achievable. It is advantageously possible to provide an analytics system, based on scripts, with an especially data-safe system architecture.

By the analytics system being “data protection safeguarding” is in particular to be understood that the analytics system does not offer any (intended and unintended) paths via which sensitive data, e. g. patient data, or other data from which deductions could be made to individual persons, could leave a protected space (the analytics environment, respectively the hospital information system) which the analytics system communicates with. Federated patient data are in particular patient data preserved in a federated manner, preferably patient data preserved by different analytics environments in different locations. Federated patient data are in particular stored in federated patient databases (in the individual patient databases of the individual analytics environments) and cannot be changed in these patient databases by the analytics system. In particular, neither the federated patient data nor portions of the federated patient data or copies thereof leave the local analytics environments, preferably the hospital information systems. Preferably only aggregated data/reports/results, like for example statistics, can leave the local analytics environments, preferably the hospital information systems. The local analytics environments may be health information systems/hospital information systems (HIS) or parts of health information systems/hospital information systems of different health institutions/hospitals. These health information systems/hospital information systems are usually fixedly assigned to respectively one health institution/one hospital and are secured against unauthorized access from outside. The analytics environments may be realized as part of an intranet, in particular of the health institutions/hospitals, or may preferably be arranged in intranets, in particular of the health institutions/hospitals. Preferably these intranets are protected against access from outside, e. g. from the Internet, for example by physical separation from the Internet or by data safety-related provisions, like firewalls or the like. Preferentially each of the analytics environments that are arranged locally in the intranets is realized as a software-technical and/or hardware-technical module installed in the respective health information system/hospital information system. It is for example conceivable that the respective analytics environment is embodied as a separate server integrated in the respective intranet, or that the respective analytics environment is embodied as a software module installed on an existing server of the respective intranet. A possible path for an installation of the analytics environment is an installation on a server provided in the respective health institution/hospital.

A “spatial separation” is in particular to mean a geographical separation, which preferably amounts at least to several kilometers. In particular, the spatial separation is to mean an arrangement in several different hospitals/hospital intranets. A “system-wise separation” is in particular to mean an arrangement in/assignment to different, preferably non-connected, health information systems/hospital information systems, preferably their intranets. In particular, each local patient analytics database comprises only a portion of the federated patient data which the analytics system works with. Herein the local patient analytics databases are preferably at least substantially free of overlap. However, in some cases there may be overlaps, for example if patients are treated in two hospitals at the same time. The local patient analytics database preferably comprises only anonymized and/or de-identified patient data. The anonymized and/or de-identified patient data comprised in the local patient analytics database are generated from original federated patient data stored in the hospital information systems/health information systems. For example, the respective local analytics environment, preferably a local data extraction module of the local analytics environment, obtains the original federated patient data from the patient dossiers arranged in the same hospital information system/health information system as the respective analytics environment, anonymizes and/or de-identifies them (for example by means of an anonymization module of the respective analytics environment) and then stores them in the local patient analytics database of the respective analytics environment in such a way that they are available for analytics processes of the local analytics module. The respective local analytics module is preferably realized so as to be at least partly integrated in the respective analytics environment. In particular, the respective local analytics module may at least partly use hardware of a computer system of the respective analytics environment, for example a processor or a memory, and/or may share a processor or a memory with other modules of the respective analytics environment. Alternatively, the respective local analytics module may be realized at least partly as a separate server within the computer system of the analytics environment and/or may be implemented within the intranet of the respective hospital information system. “Configured” is in particular to mean specifically programmed, designed and/or equipped. By an object being configured for a certain function is in particular to be understood that the object fulfils and/or carries out said certain function in at least one application state and/or operation state. It is conceivable that several intranets are joined to form a cloud-based extranet within the same country, which is for example realized in the Brazilian health system.

A “script” is in particular to mean a text document comprising orders which are comprehensible for a computer. For example, the script, in particular a source code of the script, may be written in the programming language Python, in the programming language R, in the programming language Java or in a further programming language. Scripts are in particular to be understood as program instructions to be carried out with the patient data, in particular with the respectively locally available portions of the federated patient data. Simple scripts may, for example, count a frequency of certain patient facts within the respectively locally available patient data. More comprehensive scripts could, for example, determine relations between facts (e. g. a change from first-line treatment to second-line treatment) on the basis of the respectively locally available patient data. More comprehensive scripts could, for example, filter out patterns or treatment sequences/diagnosis sequences (e. g. “first medication 1, then medication 2” or “first diagnosis X, then treatment Y”, etc.). Complex scripts could, for example, comprise instructions for (locally) generating models for machine learning, which are preferably intended to be applied to the respectively locally available patient data.

The external operator and/or programming interface system is preferably embodied as a central dedicated server offering an access option for users (for example operators and/or script programmers) of the analytics system, for example scientists, pharmacy companies, etc. Alternatively, the external operator and/or programming interface system may be embodied as a distributed system, for example as a cloud computing system. Preferably the external operator and/or programming interface system is arranged outside any hospital information systems/health information systems and/or their intranets. In particular, the external operator and/or programming interface system provides a user interface for creating scripts and/or for uploading already created scripts. In particular, the external operator and/or programming interface system is configured to provide the scripts for retrieval by the analytics environment(s). In particular, the external operator and/or programming interface system is not capable of sending the scripts to the analytics environment(s) without a request or on its own initiative or upon a user command. In particular, the analytics environments do not allow building an inbound connection that is initiated by the external operator and/or programming interface system. In particular, the analytics environments block any attempts of the external operator and/or programming interface system to build inbound connections to the analytics environments, which are initiated by the external operator and/or programming interface system.

In particular, the analytics environments are free of inbound ports and exclusively have outbound ports, thus advantageously ensuring that connections between the external operator and/or programming interface system and the analytics environments can always be initiated only from the side of the analytics environments. This advantageously allows achieving that the entire data transfer from the external operator and/or programming interface system is completely transparent for the side of the analytics environments. Moreover, unauthorized access to the analytics environments via the external operator and/or programming interface system, for example by “hacking” can advantageously be made impossible. An “outbound connection” is in particular to mean a connection between at least a portion of at least one of the analytics environments and the external operator and/or programming interface system, which can be initiated exclusively from the side of the analytics environments. In particular, a data transport between at least a portion of at least one of the analytics environments and the external operator and/or programming interface system by an outbound connection is controllable exclusively by the side of the analytics environments. In particular, data transport units of the analytics environments are free of open ports. In particular, the external operator and/or programming interface system can send exclusively data to the analytics environments which were previously requested by at least one of the analytics environments. Preferably the data transfer between the analytics environments and the external operator and/or programming interface system is free of deanonymized datasets, in particular deanonymized patient datasets. Preferably the data transfer between the analytics environments and the external operator and/or programming interface system is free of non-aggregated anonymized datasets, in particular anonymized patient datasets, which originally described individual persons. Preferably the data transfer between the analytics environments and the external operator and/or programming interface system is free of identifiers and/or of features by which a person and/or a patient could be identifiable, in particular while observing data protection regulations and laws. In particular, the data transfer from the analytics environments to the external operator and/or programming interface system is limited to aggregated data/reports/results, which are free of anonymized or deanonymized patient data that can be associated with individual persons.

In particular, the analytics environments are configured to transmit/make available their analytics results to the external operator and/or programming interface system only as aggregated data/as an aggregated report/as aggregated results (e. g. numbers of hits and/or hit categories). In particular, the analytics environments are configured to transmit their obtained analytics results only in the form of data from which no information at all is retrievable by which a person and/or a patient could be identified.

It is further proposed that at least a large portion of the analytics environments, in particular all analytics environments located within at least one country, for example the USA, Germany, France or Switzerland etc., preferably all existing analytics environments, are arranged within specially secured and/or access-restricted regions, for example intranets, of health information systems, in particular hospital information systems, and that the operator and/or programming interface system is arranged outside these specially secured and/or access-restricted regions of health information systems, in particular hospital information systems. In this way, advantageously a high level of data safety is achievable, providing at the same time centralized searchability. It is in particular possible to provide a particularly advantageous system architecture for analytics systems, which combines a high level of data safety and high user-friendliness. In particular, a particularly advantageous system architecture for analytics systems can be provided, allowing (centralized) utilization of scripts for data analytics and at the same time ensuring maximal data safety. “A large portion” is to mean in particular 85%, preferably 90%, preferentially 95% and particularly preferably 99%. Especially advantageously all analytics environments are arranged within specially secured and/or access-restricted regions, e. g. intranets, of health information systems, in particular hospital information systems; an arrangement of individual analytics environments, which has a workaround of a protection scope of a patent as its prevailing aim and does not involve any essential technical advantages, shall not be understood as a deviation from the term “all”.

Moreover, it is proposed that the analytics environments in each case comprise at least one script retrieval module, which is configured to carry out an, in particular regular, query of the external interface system for new scripts provided by the external operator and/or programming interface system for the benefit of the local analytics modules. This advantageously allows providing especially high system safety, in particular an especially safe system architecture. Advantageously only those scripts can enter the analytics environments which are retrieved in a targeted and intended manner by the script retrieval modules of the analytics environments. It is advantageously possible to prevent undesired/malicious scripts from entering the analytics environments. In particular, the script retrieval module only looks in one or several known and/or trustworthy places for new scripts to be downloaded by the respective analytics environment. In particular, the scripts can be provided for a selection of all analytics environments or for retrieval by all analytics environments. The selection of the analytics environments for this may be notified, for example, by metadata or by selecting defined providing places/providing paths (“points”), which are searched only by certain analytics environments. Preferentially each of the script retrieval modules is realized as a software-technical and/or hardware-technical module installed in the respective health information system/hospital information system. The respective script retrieval module is preferably realized so as to be at least partly integrated in the respective analytics environment. In particular, the respective script retrieval module may at least partly use a hardware of a computer system of the respective analytics environment, e. g. a processor or a memory, and/or may at least partly share such hardware with other modules of the respective analytics environment. Alternatively, the respective script retrieval module may be at least partly implemented within the computer system of the analytics environment as a separate server and/or may be at least partly implemented within the intranet of the respective hospital information system. In particular, the query by the script retrieval module is realized without request, and preferably without an outside influence, in particular without an influence by the external operator and/or programming interface system. The query may be carried out, for example, periodically, e. g. every minute, every hour, every day, etc. The query period is herein preferably configurable. It is however also conceivable that a query can be forced manually from the side of one or several analytics environment(s). It is conceivable that the script retrieval module always retrieves all the scripts provided or that it makes a comparison pre-download and retrieves only those scripts which have not been present in the analytics environment before. Preferentially the script retrieval module always only downloads the new scripts which were not present in the analytics environment before. If the query shows that new scripts have been provided for retrieval, download of the scripts to the analytics environment(s) is realized via the connections initiated by the respective analytics environments, preferably based on outbound connections.

If the script retrieval module is then configured, when finding a newly provided script, to induce a download of the provided script from the external interface system by means of the connections, in particular the outbound connection, initiated from the analytics environments, it will advantageously be possible to provide particularly high system safety, in particular an especially safe system architecture. Advantageously only such scripts can enter the analytics environments which were retrieved in a targeted and intended manner by the script retrieval modules of the analytics environments. It is advantageously possible to prevent entrance of undesired/malicious scripts in the analytics environments.

It is further proposed that the analytics environment includes a local program library database comprising program libraries, e. g. Python program libraries or program libraries of other programming languages, and/or auxiliary software modules which can be executed by the scripts, and that a script-initiated execution and/or downloading, e. g. via the Internet, of external program libraries and/or auxiliary software modules not comprised in the local program library database is prevented within the analytics environment. This advantageously allows providing particularly high system safety, in particular an especially safe system architecture. Advantageously only such program libraries and/or auxiliary software modules can be executed in the analytics environment, e. g. by the local analytics module, which were previously subjected to a safety check. Advantageously complete control and/or transparency are/is achievable of all program libraries and/or auxiliary software modules executed within the analytics environment. It is advantageously possible to prevent undesired/malicious program libraries and/or auxiliary software modules from entering the analytics environments and/or from being executed by the analytics environment/the local analytics module. In particular, each of the program libraries of the local program library databases is assigned a version number. In particular, each of the program libraries is installed locally with the corresponding version number. Preferably, for an actualization of a program library the version number is matched. Preferably, scripts accessing a program library must be compatible with the currently installed/valid version number of the program library.

If the analytics environments in each case comprise at least one library retrieval module, which is configured to carry out an, in particular regular, query of the external operator and/or programming interface system for new external program libraries and/or auxiliary software modules provided, in particular authorized, for the local program library database by the external operator and/or programming interface system; and in particular, when a newly provided, in particular authorized, external program library and/or auxiliary software module has been found, to induce a download—preferably from the external operator and/or programming interface system—of the provided program library and/or auxiliary software module by means of the connections, preferably the outbound connections, that are initiated from the analytics environments, it will advantageously be possible to provide particularly high system safety, in particular an especially safe system architecture. Advantageously complete control and/or transparency is achievable of all program libraries and/or auxiliary software modules executed within the analytics environment. In particular, the library retrieval module looks only in one or several known and/or trustworthy place/s for new program libraries and/or auxiliary software modules for download by the respective analytics environment. It is in particular possible that the program libraries and/or auxiliary software modules are made available for retrieval for a selection of all analytics environments or for all analytics environments. The selection of the analytics environments for this may be notified, for example, by metadata or by selecting defined providing places/providing paths (“points”) which are searched only by certain analytics environments. Preferentially, each of the library retrieval modules is realized as a software-technical and/or hardware-technical module installed in the respective health information system/hospital information system. The respective library retrieval module is preferably realized so as to be at least partly integrated in the respective analytics environment. In particular, the respective library retrieval module may at least partly use hardware of a computer system of the respective analytics environment, e. g. a processor or a memory, and/or may share such hardware with other modules of the respective analytics environment. Alternatively, the respective library retrieval module may be implemented at least partly as a separate server within the computer system of the analytics environment and/or within the intranet of the respective hospital information system. In particular, the query by the library retrieval module is made without request, and preferably without any outside influence, in particular without an influence by the external operator and/or programming interface system. The query may be made, for example, periodically, e. g. every minute, every hour, every day, etc. It is however also conceivable that a query can be forced manually from the side of one or several analytics environment/s, or that a query is triggered by the execution of a script trying to access a program library that is not yet present locally and/or to access an auxiliary software module that is not yet present locally. It is conceivable that the library retrieval module always retrieves all provided program libraries and/or auxiliary software modules, or that it does a comparison pre-download and retrieves only those program libraries and/or auxiliary software modules which were not present in the analytics environment before or which are currently requested by a script. If the query finds that new program libraries and/or auxiliary software modules have been made available for retrieval, the download of the program libraries and/or auxiliary software modules to the analytics environment/s is realized via the connections, preferably outbound connections, initiated from the respective analytics environments. It is conceivable that the auxiliary software modules and/or program libraries provided in the retrieval points have been subjected in advance to a check, in particular a safety check. Preferably, only those auxiliary software modules and/or program libraries are provided for the analytics environments to find which have passed a safety check beforehand. The safety check may be carried out in an automated manner by the external operator and/or programming interface system or manually. Preferably, by the external operator and/or programming interface system only explicitly released auxiliary software modules and/or program libraries are provided such that they can be found by the library retrieval modules. In particular, the analytics environments include provisions for making an installation of an unauthorized program library or of an unauthorized auxiliary software module in the analytics environments impossible.

Beyond this it is proposed that the external operator and/or programming interface system comprises a script-testing environment, which is configured for testing new scripts or scripts still in development—in particular using artificial test environment patient data provided by the external operator and/or programming interface system—before said scripts are made available for a transfer to the analytics environment/s. This advantageously allows achieving a high level of user-friendliness. Advantageously, a high analytics quality, in particular a high quality of the results of the analytics system, is attainable. The script-testing environment is in particular accessible for external users (located outside the intranet) via a user interface provided by the external operator and/or programming interface system. The external operator and/or programming interface system has access to the artificial test environment patient data, which cannot be associated with real persons/patients. The artificial test environment patient data may hence be stored on the external operator and/or programming interface system.

It is also proposed that the scripts that can be executed by the local analytics module may be analytics scripts for a purely algorithmic, in particular ML-independent, evaluation of those portions of the federated patient data which are accessible by the local analytics module (cf. for example the aforementioned simple and more comprehensive scripts. This advantageously enables optimization of an analytics result. Advantageously, in comparison to pure query systems, considerable improvement of a quality of the analytics results and significant increase of the available analytics possibilities are enabled.

Alternatively or in addition thereto, it is proposed that the scripts that can be executed by the local analytics module may be scripts for creating models for machine learning (c.f. for example the aforementioned complex scripts), which are in particular configured for a machine-learning-based evaluation of those portions of the federated patient data which are accessible by the local analytics module. This advantageously enables optimization of an analytics result. Advantageously, in comparison to pure query systems or to the analytics scripts, considerable improvement of a quality of the analytics results and significant increase of the available analytics possibilities are achievable. It is conceivable that the local analytics module is configured to execute analytics scripts and scripts for creating models for machine learning, depending on what scripts are provided to the local analytics module. If the local analytics module is embedded in a simple hardware environment, mostly or exclusively analytics scripts (the aforementioned simple and more comprehensive scripts) may be executable. If the local analytics module is embedded in a high-performance hardware environment, for example in a hardware environment having a GPU, it is possible that also scripts for creating models for machine learning (the aforementioned complex scripts) are executable. In particular, by an execution in the analytics environment such complex scripts may create a machine learning model operating in the respective analytics environment and having access to the respective local patient databases. In particular, a machine learning model of an analytics environment created in this way is also restricted locally and in particular does not communicate with machine learning models of other analytics environments. In particular, in order to safeguard high data safety, these machine learning models are not configured for so-called federated learning.

In this context it is therefore proposed that the models for machine learning are restricted to the respective analytics environment, preferably to the respective local analytics module. This advantageously allows ensuring an especially high degree of data safety. It is advantageously possible to create an especially secure machine learning approach for the evaluation of sensitive patient data. In particular, in this case only on-site training of the machine learning models will take place, and in particular any exchange between several machine learning models from different analytics environments will be foregone.

It is moreover proposed that the analytics environments in each case comprise at least one output module, which is configured for an output of analytics results of the local analytics modules, wherein any output of analytics results externally, for example to the external operator and/or programming interface system, is limited to an output to external servers that were defined beforehand, e. g. in a whitelist. This advantageously allows further increasing data safety. It is advantageously possible to prevent an output of analytics results to unknown and/or unauthorized sources. Advantageously a system architecture with an additional safety level is obtainable. It is for example possible—if a malicious script has somehow entered the analytics environment—to prevent this script from sending data to an unknown and/or unauthorized source. Advantageously, these data can then be caught on the external server. Preferentially each of the output modules is embodied as a software-technical and/or hardware-technical module installed in the respective health information system/hospital information system. The respective output module is preferably realized so as to be at least partly integrated in the respective analytics environment. In particular, the respective output module may at least partly use a hardware of a computer system of the respective analytics environment, for example a processor or a memory, and/or may share such hardware with other modules of the respective analytics environment.

Alternatively, the respective output module may be implemented at least partly as a separate server within the computer system of the analytics environment and/or within the intranet of the respective hospital information system. In particular, the external server is realized as a proxy server, in particular as a whitelisted proxy server. In particular, the external server may form part of the external operator and/or programming interface system. Alternatively, however, implementation separate therefrom is also conceivable.

If furthermore any output of analytics results externally, for example to the external operator and/or programming interface system, is limited to aggregated data/reports/results, high data safety, in particular high-grade protection of sensitive data, like patient data, is advantageously achievable. In particular, the aggregated data do not comprise anything that could permit inference to a real person/a real patient. Preferably the aggregated data comprise only numbers, (binary) yes-no statements and/or single letters, which for example indicate a category or something like that. In particular, the respective output modules transmit the aggregated data/reports/results to the external operator and/or programming interface system, in particular to an output unit and/or a dashboard of the external operator and/or programming interface system.

In an alternative analytics system, which does not belong to the core of the invention but is also conceivable, any output of analytics results externally, for example to the external operator and/or programming interface system, could be limited to aggregated data and in addition to model data/model parameters for models for machine learning, which are made unavailable for inference by means of an obscuration technique, like for example a Differential Privacy Sparse Vector Technique, a Differentially-Private Stochastic Gradient Descent Technique, or the like. In such a case, following a federated-learning approach could be enabled. The outputted model data/model parameters could then be retrieved from other analytics environments by the external operator and/or programming interface system for the purpose of generating a feedback loop, or they could be converged/averaged in the external operator and/or programming interface system. Moreover, the model data/model parameters of the respective models could be compared in order to determine cohort differences.

In addition, it is proposed that the analytics system, in particular each of the analytics environments of the analytics system, comprises local data extraction modules, which are configured to read out patient dossiers of a hospital information system and, based on these, to generate the portion of the federated patient data which is respectively allocated to the analytics environment and is accessible for the respective local analytics modules. This in particular allows providing an advantageous system architecture. The data extraction module may, for example, comprise the anonymization module. Preferentially each of the data extraction modules is embodied as a software-technical and/or hardware-technical module installed in the respective health information system/hospital information system. The respective data extraction module is preferably realized so as to be at least partly integrated in the respective analytics environment. In particular, the respective data extraction module may at least partly use a hardware of a computer system of the respective analytics environment, for example a processor or a memory, and/or may share such hardware with other modules of the respective analytics environment. Alternatively, the respective data extraction module may be implemented at least partly as a separate server within the computer system of the analytics environment and/or within the intranet of the respective hospital information system. In particular, the data extraction module stores the extracted data in the local patient database of the local analytics environment. In particular, the analytics environment has no direct access to patient dossiers of the respective hospital information system. The local data extraction module may be configured to process the extracted data and to store them in the local patient analytics database in such a way that they can be read out/processed easily by scripts, for example in the form of a table.

If the data extraction module, in particular the anonymization module, comprises an anonymization and/or de-identifying routine which is configured, during a read-out of the patient dossiers and the generation of the data allocated to the federated patient data, to remove and/or to obscure all data features that are contained in the original patient dossier and permit an allocation to an individual person, it is advantageously possible to achieve particularly high data safety, in particular by multi-level protection. Advantageously, a system architecture can be created that is especially data protection safeguarding. In particular, the data extraction module stores only such patient data in the local patient analytics database which are de-identified and/or anonymized following the anonymization and/or de-identifying routine.

Beyond this it is proposed that the analytics environments in each case comprise a local user interface, which permits on-site analytics of the locally present portion of the federated patient data and/or which enables on-site execution of the scripts on the locally present portion of the federated patient data. This advantageously allows attaining a high degree of user-friendliness. In particular, the local user interface provides a local dashboard. In particular, the local user interface is accessible exclusively via the intranet of the respective health institutions/hospitals. If applicable, a possibility of access to the local user interface may be provided via a VPN tunnel into the intranet of the respective health institutions/hospitals. In particular, the local user interface is inaccessible for external developers, in particular for creators and providers of scripts by means of the external operator and/or programming interface system.

Furthermore, the analytics environment and the operator and/or programming interface system of the analytics system are proposed, by means of which an advantageous system architecture is achievable.

Moreover, a data protection safeguarding analytics method for federated patient data, in particular for a recruiting of test persons for clinical surveys, for identifying trends, for obtaining optimization proposals for treatments, for an implementation of clinical dashboards and/or for a modeling of machine learning methods using the analytics system, is proposed, wherein the scripts that can be executed by the local analytics modules are created and/or provided via the external operator and/or programming interface system, and wherein data and scripts are transferred between the local analytics modules and the external interface system exclusively via connections, in particular outbound connections, that are initiated from the respective analytics environment. This advantageously allows providing a data protection safeguarding analytics possibility which is based on an execution of scripts.

The analytics system according to the invention, the analytics environment according to the invention, the operator and/or programming interface system according to the invention and the analytics method according to the invention shall here not be limited to the application and implementation described above. In particular, in order to fulfil a functionality that is described here, the analytics system according to the invention, the analytics environment according to the invention, the operator and/or programming interface system according to the invention and the analytics method according to the invention may comprise a number of individual elements, components, method steps and units that differs from a number given here.

### DRAWINGS

Further advantages will become apparent from the following description of the drawings. In the drawings an exemplary embodiment of the invention is illustrated. The drawings, the description and the claims contain a plurality of features in combination. Someone skilled in the art will purposefully also consider the features separately and will find further expedient combinations.

It is shown in:

FIG. 1 a schematic illustration of a comprehensive system architecture of a data protection safeguarding analytics system for federated patient data,

FIG. 2 a schematic illustration of the data protection safeguarding analytics system including an illustration of crucial steps of a data protection safeguarding analytics method for federated patient data, which applies the analytics system,

FIG. 3 an exemplary output of aggregated analytics results of an output module of the analytics system,

FIG. 4 an exemplary output of a local user interface of a local analytics environment of the analytics system, and

FIG. 5 a schematic flowchart of the data protection safeguarding analytics method using the analytics system.

### DESCRIPTION OF THE EXEMPLARY EMBODIMENTS

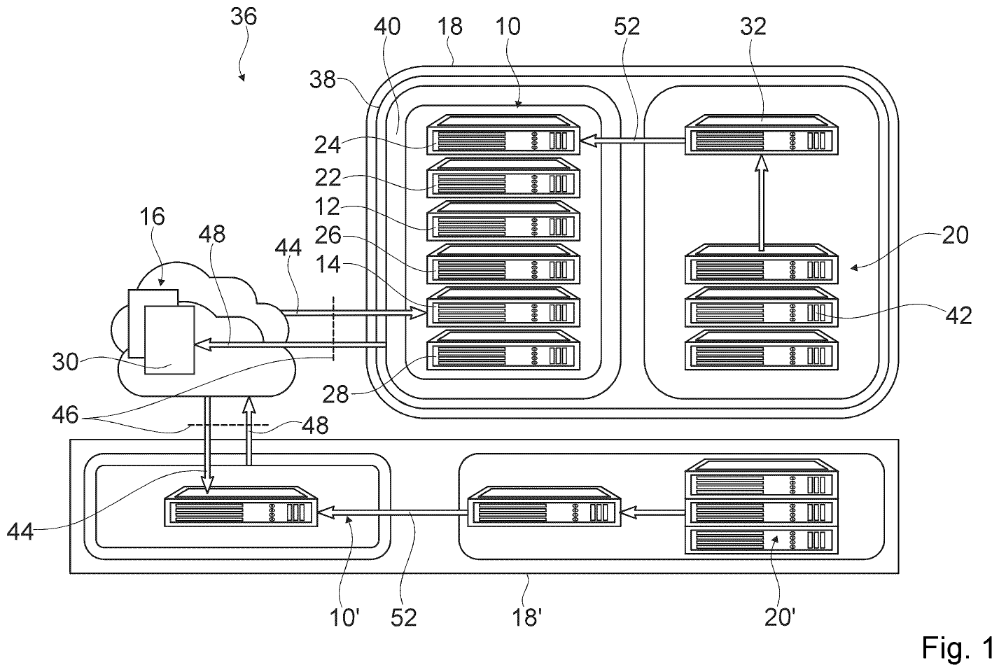

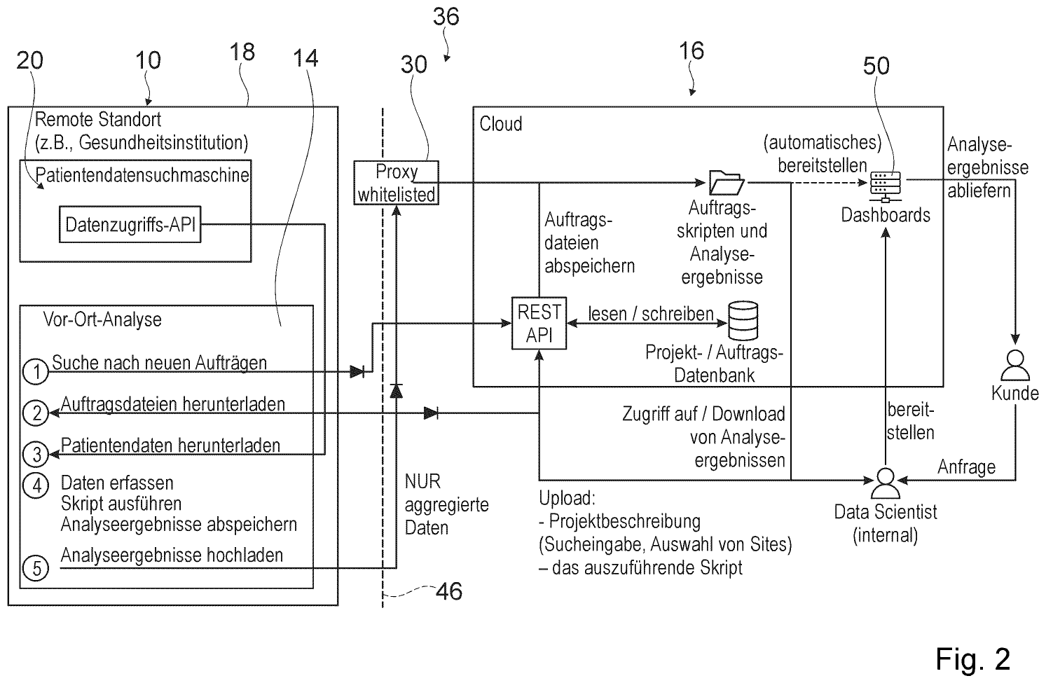

FIGS. 1 and 2 respectively show schematic illustrations of a data protection safeguarding analytics system 36 for federated patient data. The analytics system 36 comprises a plurality of analytics environments 10, 10′. By way of example, two analytics environments 10, 10′ are shown in FIG. 1; however, considerably more than two analytics environments 10, 10′ may participate in the analytics system 36. The analytics environments 10, 10′ are arranged so as to be spatially separate from one another. The analytics environments 10, 10′ are separate from each other system-wise. The analytics environments 10, 10′ of the analytics system 36 are in each case arranged within specially secured and/or access-restricted regions 18, 18′ of health information systems 20, 20′. The specially secured and/or access-restricted regions 18, 18′ are in each case realized by intranets of the respective health information systems 20, 20′. The health information systems 20, 20′ may in each case be realized as hospital information systems (HIS) or as other information systems of other health institutions. The specially secured and/or access-restricted region 18 is protected by a health information system firewall 38. The analytics environment 10 further comprises a further analytics environment firewall 40, which is configured for a protection of the analytics environment 10. The modules and components of the analytics environment 10 are thus situated behind at least two firewalls 38, 40, the health information system firewall 38 and the analytics environment firewall 40. The health information system 20, 20′ is free of ports open for the analytics environments 10, 10′, in particular free of VPN ports or inbound connection ports open for the analytics environments 10, 10′. In FIG. 2 a separation line is depicted, which is to indicate a border between a specially secured and/or access-restricted region 18, 18′, respectively an intranet (here: to the left of the separation line 46) and a publicly accessible region, respectively the Internet.

The health information system 20, 20′ comprises a patient dossier database 42. The patient dossier database 42 is arranged outside the analytics environment 10, 10′. The analytics environment 10, 10′ has no access authorization for an access to the patient dossier database 42 of the respective health information system 20, 20′. In the patient dossier database 42 the original patient dossiers of the health institution are stored. These patient dossiers comprise highly sensitive data. The analytics environments 10, 10′ in each case comprise a local patient analytics database 12. The local patient analytics databases 12 are configured to respectively comprise, in particular memorize, a portion of the federated patient data which can be evaluated by the analytics system 36. The federated patient data memorized in the patient analytics databases 12 do not correspond to the patient dossiers of the respective health information system 20, 20′. The federated patient data memorized in the patient analytics databases 12 have been generated only on the basis of the patient dossiers of the respective health information system 20, 20′.

The analytics environments 10, 10′ in each case comprise a local analytics module 14. The local analytics modules 14 are configured at least for an execution of scripts on the portion of the federated patient data that is available in the respective local patient analytics database 12. The local analytics modules 14 may furthermore be configured for a simple searching, based on simple search strings, of the portions of the federated patient data available in the respective local patient analytics databases 12. The scripts which can be executed by the local analytics module 14 may be analytics scripts for a purely algorithmic (machine-learning-independent) evaluation of those portions of the federated patient data which the local analytics module 14 has access to. Beyond this, the scripts that can be executed by the local analytics module 14 may be scripts for creating models for machine learning. The scripts for creating models for machine learning are configured for locally creating machine learning models in the analytics environments 10, 10′. The scripts for creating models for machine learning are configured for a machine-learning-based evaluation of those portions of the federated patient data which the local analytics module 14 has access to. The models for machine learning created locally by the scripts are limited to the respective local analytics module 14.

The analytics system 36 comprises a local data extraction module 32 in each of the health information systems 20, 20′. It is conceivable that the local data extraction module 32 is in each case assigned to the analytics environment 10, 10′ of the respective health information system 20, 20′. Alternatively, however, as is shown exemplarily in FIG. 1, the local data extraction module 32 may be an interface module, arranged outside the analytics environment 10, 10′, between the patient dossier database 42 of the health information system 20, 20′ and the analytics environment 10, 10′ of the health information system 20, 20′. The local data extraction module 32 is configured for reading out the patient dossiers of the respectively allocated health information system 20, 20′ and, based thereon, to generate the portion of the federated patient data which is respectively assigned to the analytics environment 10, 10′, which the respective local analytics modules 14 have access to and which is memorized in the local patient analytics database 12. The local data extraction module 32 may be realized as a local ETL (Extract-Transfer-Load) server. The data extraction module 32 may be realized as an encrypted server. The local data extraction module 32 comprises an anonymization and/or de-identifying routine. The anonymization and/or de-identifying routine of the data extraction module 32 is configured, during the reading-out of the patient dossiers from the patient dossier database 42 and the generation of the data assigned to the federated patient data/of the data stored in the patient analytics database 12 of the analytics environment 10, 10′, to remove (e. g. by de-identifying) and/or to obscure (e. g. by anonymization) all data features contained in the original patient dossier which permit an allocation to an individual person. The local data extraction module 32 is configured to transfer the de-identified and/or anonymized patient data to the patient analytics database 12 and to store them thereon (cf. arrow 52).

The analytics system 36 comprises an interface system 16. The interface system 16 forms an operator and/or programming interface system 16. The interface system 16 is an external interface system 16, which is realized and arranged so as to be separate spatially and system-wise from each analytics environment 10, 10′ of the analytics system 36. The external operator and/or programming interface system 16 is arranged outside the specially secured and/or access-restricted regions 18 of the health information systems 20, 20′. The external operator and/or programming interface system 16 is arranged outside the intranets of the health information systems 20, 20′. The external operator and/or programming interface system 16 may be arranged and realized centrally. In the exemplary embodiment illustrated in FIG. 1, the operator and/or programming interface system 16 is realized as a distributedly arranged, cloud-based system.

The external operator and/or programming interface system 16 is configured to provide to the analytics environments 10, 10′ the scripts from an outside that are executable by the local analytics modules 14. The external operator and/or programming interface system 16 makes the scripts that are executable by the local analytics modules 14 available for a download by the analytics environments 10, 10′ in/at known and trustworthy places/addresses. The external operator and/or programming interface system 16 enables external generation of the scripts that can be executed by the local analytics modules 14. The external operator and/or programming interface system 16 comprises an operator and/or programmer interface which enables generation and/or selection of the scripts that are to be made available. The external operator and/or programming interface system 16 comprises a script-testing environment. The script-testing environment is configured for testing new scripts or scripts that are in development before they are made available for a transfer to the analytics environment/s 10, 10′. For this purpose, the external operator and/or programming interface system 16 comprises artificial test environment patient data which are intended for the testing of the scripts.

The external operator and/or programming interface system 16 is with regard to data transfer connected to each of the analytics environments 10, 10′ exclusively via connections that are initiated from the analytics environments 10, 10′. The data transfer between the external operator and/or programming interface system 16 and the analytics environments 10, 10′ takes place exclusively via connections that are initiated from the analytics environments 10, 10′. The only possible connections between the external operator and/or programming interface system 16 and the analytics environments 10, 10′ are outbound connections that are initiated from the analytics environments 10, 10′.

The analytics environments 10, 10′ in each case comprise at least one script retrieval module 22. The script retrieval module 22 is configured to carry out an, in particular regular, query of the operator and/or programming interface system 16 for new scripts provided for the local analytics modules 14 by the external operator and/or programming interface system 16. The script retrieval module 22 is configured, when finding newly provided scripts, to induce a download of the provided script/s from the external operator and/or programming interface system 16 by means of the connections, in particular the outbound connections, initiated from the analytics environments 10, 10′.

The analytics environments 10, 10′ in each case comprise a local program library database 24. The local program library databases 24 contain program libraries and/or auxiliary software modules which can be executed by scripts. The analytics environments 10, 10′ comprise software-technical and/or hardware-technical provisions which prevent script-initiated execution and/or downloading of external program libraries and/or auxiliary software modules that are not comprised in the local program library database 24. The analytics environments 10, 10′ comprise software-technical and/or hardware-technical provisions which prevent execution and/or downloading of external program libraries and/or auxiliary software modules from the Internet, in particular from unknown places and/or addresses that are not allocated/assigned to the external operator and/or programming interface system 16. The analytics environments 10, 10′ in each case comprise at least one library retrieval module 26. The library retrieval module 26 is configured to carry out an, in particular regular, query of the external interface system 16 for new external program libraries and/or auxiliary software modules provided, in particular authorized, for the local program library database 24 by the external interface system 16. The library retrieval module 26 is configured, when finding a newly provided, in particular authorized, external program library and/or auxiliary software module, to induce a download of the provided program library and/or auxiliary software module, preferably from the external interface system 16 and/or from a trustworthy and/or known place or address, by means of the connections, preferably the outbound connections, that are initiated from the analytics environments 10, 10′. In FIG. 1 the download of scripts, program libraries and/or auxiliary software modules from the operator and/or programming interface system 16 to the analytics environments 10, 10′ is illustrated by an arrow 44. The communication path illustrated by the arrow 44 is limited to the outbound connections. This is clarified in FIG. 2 by using diode switch symbols. Communication paths given diode switch symbols in FIG. 2 are one-way communication paths in which the target side (the side which the vertex of the triangle of the diode switch symbol points to, that is the side of FIG. 2 situated to the right of the separation line 46) is not able to initiate a communication. Initiation of a communication will in such a case always originate from the start side (the side situated opposite the vertex of the triangle of the diode switching symbol, that is the side of FIG. 2 situated to the left of the separation line 46).

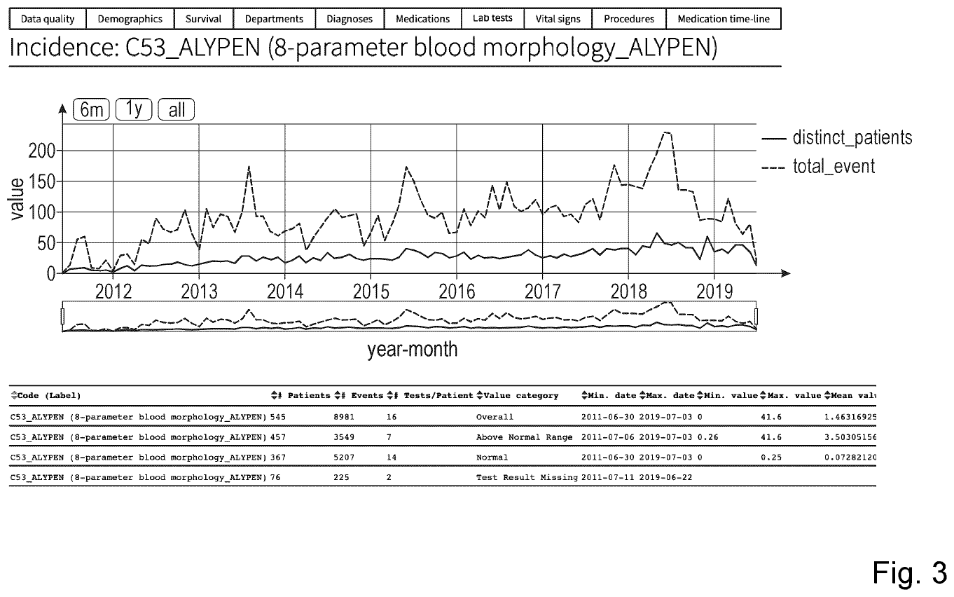

The analytics environments 10, 10′ in each case comprise an output module 28. The output modules 28 are configured for an output of analytics results of the local analytics modules 14. The output modules 28 are configured for an output of the analytics results generated by the local analytics modules 14 using the scripts. The analytics system 36 comprises at least one external server 30. The external server 30 is embodied as a proxy server. The output modules 28 are configured to restrict any output of analytics results towards an outside, for example to the external operator and/or programming interface system 16, to an output only to the externals servers 30 that were defined beforehand. For this purpose, the output modules 28 have a whitelist. The whitelist contains all external servers 30 to which transmission of analytics results of the analytics modules 14 is possible. Any output of analytics results towards an outside, for example to the external operator and/or programming interface system 16, is entirely restricted to aggregated data. The output modules 28 are configured to restrict any output of analytics results towards an outside, for example to the external operator and/or programming interface system 16, exclusively to aggregated data. In FIG. 1 the output of the analytics results is illustrated by an arrow 48. Via the path of the arrow 48, only aggregated data, which do not permit any inference to real patients, leave the analytics environments 10, 10′, respectively the health information systems 20, 20′. In FIG. 3 an exemplary output of aggregated analytics results of the output module 28 is shown. This output can be queried from an outside, e. g. via the Internet, for example via a central dashboard 50 of the operator and/or programming interface system 16 (cf. FIG. 2).

The analytics environments 10, 10′ in each case comprise a local user interface (cf. FIG. 2). The local user interface is embodied as a local dashboard. The local user interface permits on-site analytics of the portion of the federated patient data that is present locally in the respective patient analytics database 12. The local user interface permits on-site execution of the scripts on the portion of the federated patient data that is present locally in the patient analytics database 12. In FIG. 14 an exemplary output of the local user interface is illustrated, representing a data entry from the patient analytics database 12, which is allocated to a de-identified patient. This indication is only possible within the secured region 18, 18′ of the respective health institution.

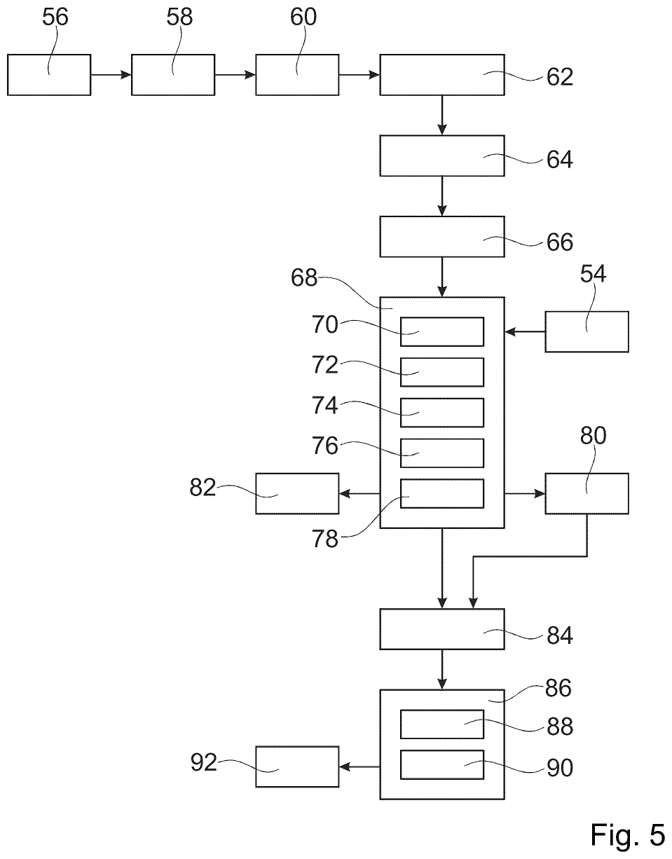

FIG. 5 shows a schematic flowchart of a data protection safeguarding analytics method for federated patient data using the analytics system 36. In at least one method step 54, by means of the respective local data extraction modules 32, patient data from the patient dossiers of the health information systems 20, 20′ are copied and modified and are stored in the local patient analytics databases 12 in a modified format. Herein the patient data are modified in the method step 54 by means of the anonymization and/or de-identifying routine in such a way that all data features contained in the original patient dossier which permit inference to an individual person are removed and/or obscured. As a result, in each case only anonymized and/or de-identified patient data from the health information system 20, 20′ allocated to the respective patient analytics database 12 are memorized in the individual local patient analytics databases 12. The anonymized and/or de-identified patient data present in the local patient analytics databases 12 are then available for the analytics by the analytics modules 14. In a method step 56, a script is created outside the analytics environments 10, 10′ and outside the health information systems 20, 20′. The script may be an analytics script or a script for creating models for machine learning. The script may be generated by means of the external operator and/or programming interface system 16 or may be transferred to the external operator and/or programming interface system 16. In at least one method step 58, the script that has been generated or received is tested in the script-testing environment of the external operator and/or programming interface system 16 using the artificial test environment patient data. In at least one further method step 60, the script is subjected to a manual or automated safety check.

In at least one further method step 62, the—preferably safety-checked—script is kept in readiness for download by the analytics environments 10, 10′ in a place/at an address designated for this purpose. For this a selection can be made from the total number of analytics environments 10, 10′, corresponding to a selection of health institutions that are to be comprised in the analytics. In at least one method step 64, the local script retrieval modules 22 query the designated places/addresses of the external operator and/or programming interface system 16 in order to find out whether one or several script/s has/have been provided for the analytics environment 10, 10′ allocated to the respective local script-retrieval module 22. This may be realized, for example, via one-way communication of the respective analytics environment 10, 10′ with a REST API of the external operator and/or programming interface system 16. In at least one further method step 66, the provided scripts are downloaded from the external operator and/or programming interface system 16 by the script retrieval modules 22 of the respective analytics environments 10, 10′. In at least one further method step 68, the downloaded scripts are executed by the respective analytics environments 10, 10′. By the execution of the scripts, analytics processes of the patient data memorized in the local patient analytics databases 12 are carried out. By the execution of the scripts the analytics results are obtained. It is conceivable that the scripts make use of program libraries or auxiliary software modules which are not part of the script but are intended to be executed by the script.

In a method substep 70 of the method step 68, at least one program library and/or at least one auxiliary software module from the local program library database 24 of the respective analytics environment 10, 10′ is used. This can be done only if the respectively required program library and/or the respectively required auxiliary software module is present in the local program library database 24. If in method step 68 a script attempts to access a program library and/or an auxiliary software module that is only externally available, the analytics environment 10, 10′ blocks this attempt and/or searches for the desired program library and/or the desired auxiliary software module in a designated trustworthy place/address of the external operator and/or programming interface system 16. References of scripts to further places or addresses where program libraries and/or auxiliary software modules could be found are completely blocked by the analytics environments 10, 10′. In a further method substep 72 of the method step 68, new program libraries and/or auxiliary software modules are subjected to a safety check before they are released for the analytics environments 10, 10′. In a further method substep 74 of the method step 68, the safety-checked new program libraries and/or auxiliary software modules are made available in a place/at an address of the external operator and/or programming interface system 16 that is designated for this purpose. In a further method substep 76 of the method step 68, the local library retrieval modules 26 query the designated places/addresses of the external operator and/or programming interface system 16 in order to find out if one or several authorized program libraries and/or auxiliary software modules have been made available in/at said designated places/addresses. In at least one further method substep 78 of the method step 68, the provided program libraries and/or auxiliary software modules are downloaded from the external operator and/or programming interface system 16 by the library retrieval modules 26 of the respective analytics environments 10, 10′. As soon as the downloaded program libraries and/or auxiliary software modules have been stored in the respective local program library database 24, they are locally available for use by the scripts. In at least one method step 80, a script—if it is realized as a script for creating a model for machine learning—may create and activate a machine learning model, which is restricted to the analytics environment 10, 10′.

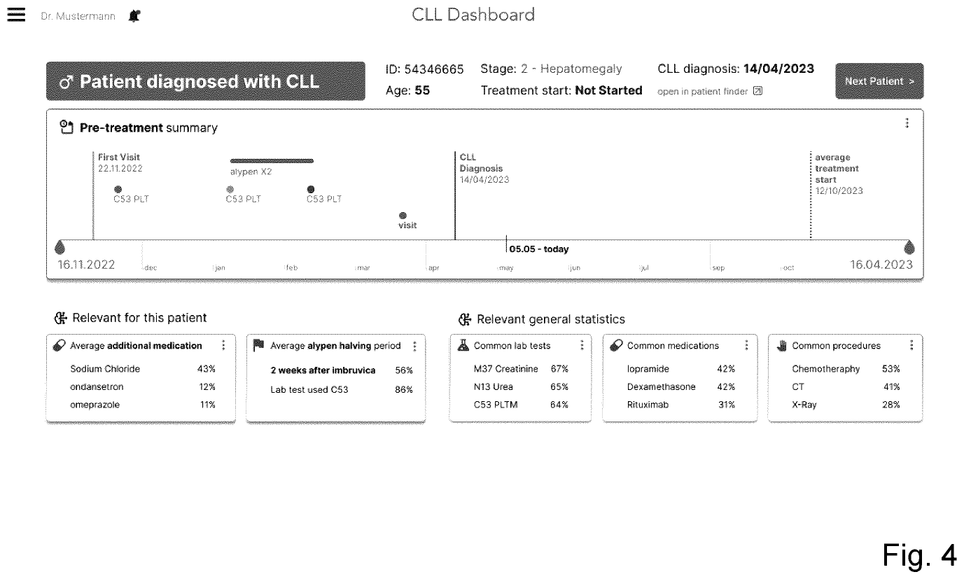

In at least one method step 82, a local dashboard (cf. FIG. 4), which permits on-site viewing of the analytics results in a non-aggregated format, is called up via the local user interface by a user who is localized in the location of the respective health institution. For this the user needs a special authorization (e. g. a password), which enables him to access the protected analytics environment 10, 10′.

In at least one method step 84, an aggregated analytics result of the analytics, initiated by the script, of the patient data stored in the patient analytics database 12 is generated in the respective analytics environments 10, 10′ using the analytics modules 14. In at least one method step 86, the aggregated analytics result is outputted by the respective local output module 28. For this first of all, in a method substep 88 of the method step 86, a check is made whether the place/address to which the output module 28/the script wants to send the analytics results is listed on a whitelist of the analytics environment 10, 10′. In the whitelist trustworthy places/addresses are listed, which may be part of the external operator and/or programming interface system 16 or which may be embodied as external servers 30, for example proxy servers, that are realized separately from the external operator and/or programming interface system 16. In a further method substep 90 of the method step 86, depending on whether the intended receiving place/receiving address for the analytics results is contained in the whitelist or not, the output of the analytics results by the output module 28 is allowed or blocked. In at least one further method step 92, the aggregated analytics results are made available to external users of the operator and/or programming interface system 16, for example end customers or scientists, via the central dashboard 50. The central dashboard 50 has for this purpose access to the external server 30 which the aggregated analytics results were transmitted to. In the data protection safeguarding analytics method data and scripts are transferred between the local analytics modules 14 and the external operator and/or programming interface system 16 exclusively via connections, in particular outbound connections, that are initiated from the respective analytics environment 10, 10′.

### REFERENCE NUMERALS

10 analytics environment
12 local patient analytics database
14 local analytics module
16 external interface system
18 region
20 health information system
22 script retrieval module
24 program library database
26 library retrieval module
28 output module
30 external server
32 local data extraction module
36 analytics system
38 health information system firewall
40 analytics environment firewall
42 patient dossier database
44 arrow
46 separation line
48 arrow
50 central dashboard
52 arrow
54 method step
56 method step
58 method step
60 method step
62 method step
64 method step
66 method step
68 method step
70 method sub-step
72 method sub-step
74 method sub-step
76 method sub-step
78 method sub-step
80 method step
82 method step
84 method step
86 method step
88 method sub-step
90 method sub-step
92 method step

1. A data protection safeguarding analytics system for federated patient data, at least with a plurality of analytics environments which are separate from one another spatially as well as system-wise, which in each case include at last one local patient analytics database comprising a portion of the federated patient data, and which in each case include at least one local analytics module that is configured at least for an execution of scripts on the portion of the federated patient data available in the respective local patient analytics database, and with at least one external operator and/or programming interface system, which is in particular arranged centrally or distributedly, which is separate from the analytics environments spatially and system-wise, by means of which the scripts that can be executed by the local analytics modules can be—in particular externally—created and/or provided, and which is with regard to data transfer connected to each of the analytics environments exclusively via connections, in particular outbound connections, that are initiated from the analytics environments.

2. The analytics system according to claim 1, wherein at least a large portion of the analytics environments, in particular all analytics environments, are arranged within specially secured and/or access-restricted regions, for example intranets, of health information systems, in particular hospital information systems, and wherein the external operator and/or programming interface system is arranged outside these specially secured and/or access-restricted regions of health information systems, in particular hospital information systems.

3. The analytics system according to claim 1, wherein the analytics environments in each case comprise at least one script retrieval module, which is configured to carry out an, in particular regular, query of the external interface system for new scripts provided by the external interface system for the local analytics modules.

4. The analytics system according to claim 3, wherein the script retrieval module is configured, when finding a newly provided script, to induce a download of the provided script from the external interface system by means of the connections, in particular the outbound connections, that are initiated from the analytics environments.

5. The analytics system according to claim 1, wherein the analytics environment includes a local program library database comprising program libraries and/or auxiliary software modules which can be executed by the scripts, and that script-initiated execution and/or downloading of external program libraries and/or auxiliary software modules not comprised in the local program library database is prevented within the analytics environment.

6. The analytics system according to claim 5, wherein the analytics environments in each case comprise at least one library retrieval module, which is configured to carry out an, in particular regular, query of the external interface system for new external program libraries and/or auxiliary software modules provided, in particular authorized, for the local program library database by the external interface system, and in particular, when a newly provided, in particular authorized, external program library and/or auxiliary software module has been found, to induce a download—preferably from the external interface system of the provided program library and/or auxiliary software module by means of the connections, preferably the outbound connections, that are initiated from the analytics environments.

7. The analytics system according to claim 5, wherein the external interface system comprises a script-testing environment, which is configured for testing new scripts or scripts still in development—in particular using artificial test environment patient data provided by the external interface system—before said scripts are made available for a transfer to the analytics environment/s.

8. The analytics system according to claim 1, wherein the scripts that can be executed by the local analytics module may be analytics scripts for a purely algorithmic, in particular ML-independent, evaluation of those portions of the federated patient data which are accessible by the local analytics module.

9. The analytics system according to claim 1, wherein the scripts that can be executed by the local analytics module may be scripts for creating models for machine learning, which are in particular configured for a machine-learning-based evaluation of those portions of the federated patient data which are accessible by the local analytics module.

10. The analytics system according to claim 1, wherein the analytics environments in each case comprise at least one output module, which is configured for an output of analytics results of the local analytics modules, wherein any output of analytics results towards an outside, for example to the external interface system, is limited to an output to external servers that were defined beforehand, e. g. in a whitelist.

11. The analytics system according to claim 1, wherein the analytics environments in each case comprise at least one output module, which is configured for an output of analytics results of the local analytics modules, wherein any output of analytics results towards an outside, for example to the external interface system, is limited to aggregated data.

12. The analytics system according to claim 9, wherein the models for machine learning are restricted to the respective local analytics module.

13. The analytics system according to claim 1, comprising local data extraction modules, which are configured to read out patient dossiers of a health information system, in particular a hospital information system, and based on these, to generate the portion of the federated patient data which is respectively allocated to the analytics environment and is accessible for the respective local analytics modules.

14. The analytics system according to claim 1, wherein the analytics environments in each case comprise a local user interface, which permits on-site analytics of the locally present portion of the federated patient data and/or which enables on-site execution of the scripts on the locally present portion of the federated patient data.

15. An analytics environment or an operator and/or programming interface system of an analytics system according to claim 1.

16. A data protection safeguarding analytics method for federated patient data, using an analytics system according to claim 1, wherein the scripts that can be executed by the local analytics modules are created and/or provided via the external operator and/or programming interface system, and wherein data and scripts are transferred between the local analytics modules and the external interface system exclusively via connections, in particular outbound connections, that are initiated from the respective analytics environment.
